(19) United States
(12) Patent Application Publication
(10) Pub. No.: US 2023/0158271 A1
(43) Pub. Date: May 25, 2023

(54) A SYSTEM AND METHOD FOR IMPROVING HOLISTIC WELLNESS OF HUMANS

(71) Applicant: Abhay Wadhwa, New York, NY (US)

(72) Inventor: Abhay Wadhwa, New York, NY (US)

(21) Appl. No.: 17/917,008

(22) PCT Filed: Apr. 5, 2021
(86) PCT No.: PCT/IN2021/050338

§ 371 Date: Oct. 5, 2022

(30) Foreign Application Priority Data
Apr. 6, 2020 (IN) 202011015056

(51) Int. Cl. A61M 21/02; A61M 21/00

(52) U.S. Cl. CPC A61M 21/02; A61M 21/0094; A61M 2021/0016; A61M 2021/0027; A61M 2021/0044

(57) ABSTRACT

Disclosed herein a method for improving holistic wellness of humans, wherein said method comprises of synergizing the light and color via spectral wavelengths inducing Simultaneous Adjacent Chroma Stimulus (SACS), sound frequencies and projection art. The synergized light and color, sound frequencies and projection art provide a sensory experience that de-stresses and realigns the body, mind and soul. The corresponding system comprises of an enclosure having projection screens and multipurpose integrated apparatus, wherein said apparatus comprises of CONTENT projecting means, color and light producing means, sound producing means and geometric reflectors, wherein these are configured to project lights on to the projection screen shown through a story-telling format, wherein said light projecting means, light producing means, sound producing means, projecting screens and reflectors are relatively placed so as to optimally produce the desired response of de-stressing and breaking the vicious cycle of stress in humans.

### FIELD OF THE INVENTION

The present invention relates to a system and method for improving holistic wellness in humans. More particularly, the present invention relates to a scientific ecosystem for mind-body and harmony that balances our neural system, endocrine system and physiological system.

### BACKGROUND OF THE INVENTION

Background description includes information that may be useful in understanding the present invention. It is not an admission that any of the information provided herein is prior art or relevant to the present invention, or that any publication specifically or implicitly referenced is prior art.

The world we are living in is becoming urbanized at an unprecedented pace. Living in urban areas, especially in large cities has its advantages but also sometimes takes its toll on not just the physical, but mental well-being of its residents. Most of the urban working population live under immense stress, mainly because of competitive work environment, living in nuclear families, lack of sanitation, air pollution, respiratory problems, communicable diseases, traffic jams, accidents, and injuries. People often fail to take out time to relax and rejuvenate as a result they suffer from various lifestyle diseases like - insomnia, hypertension, stress, etc., which may further lead to various physiological and psychological problems including problems related to metabolic activity, anxiety, delirium, and dementia.

In order to resolve the above issues, people usually take the help of medications and other addictive supplements, but such solutions are not the best ways to deal with such issues and could worsen the case since these medicines are associated with side effects. Also, such issues require holistic solutions. It is known from that certain sounds, colours, and vibrations have a substantial impact on the human body and work well as a stimulant. Many inventions, based on this understanding, were made to solve physiological and psychological problems. Some of the most popular ones were treatment or healing enhancement through music therapy or visual therapy or audio-visual therapy or colour therapy where the patient is required to devote his/her time in a specially designed environment and carry out the actions as directed by the therapist/trainer. Despite all this, such therapies or treatments are not capable of targeting the entire body for holistic wellness.

Below are the details of few prior arts relevant to the present invention:

US20070287881A1 discloses a device, system, apparatus, and method for reducing stress in an individual by creating an enhanced informational spin field environment substantially surrounding the individual. Such informational spin field environment is at least partially derived from one or more dynamically produced informational spin fields wherein electromagnetic components associated with producing one or more of such dynamically produced informational spin fields are blocked from propagating therewith, such one or more dynamically produced informational spin fields being then conducted without accompanying electromagnetic signals to the environment substantially surrounding the individual. therewith, such one or more dynamically produced informational spin fields being then conducted without accompanying electromagnetic signals to the environment substantially surrounding the individual. therewith, such one or more dynamically produced informational spin fields being then conducted without accompanying electromagnetic signals to the environment substantially surrounding the individual. therewith, such one or more dynamically produced informational spin fields being then conducted without accompanying electromagnetic signals to the environment substantially surrounding the individual.

US7141028B2 discloses a system, apparatus, and method for therapeutic light and sound, comprising the steps of exposing a user to spectral light and laterally directed sound and adjusting the manner of exposure sufficient to elicit a desired autonomic nervous response.

WO2015022697A discloses a wellness/multi-therapy system comprising: an enclosed housing having means for desired posture based of a subject/individual to be exposed to said wellness/ therapy comprising: a pyramid shaped top adapted to selectively deflect any type of cosmic and/or electromagnetic radiations that fall on its apex to the bottom through the sides while the inner center region of said pyramid is filled with safe and powerful bio energy and generate positive rich energy inside which cooperatively enables wellness/ therapy on said subject/ individual, involving selective wellness/therapy means comprising anyone or more of aroma means for aroma therapy, light and/or crystal/stone means for chakra/7 energy vortices balancing therapy, oxygen supply means for oxygen therapy with each said wellness/ therapy means being adapted to be controlled and/or programmed independently and/or in combination based on the requirement of the subject/individual in said housing. The system is adapted for faster maintenance of desired balance between spirit-mind-body together in a harmonious way in shortest possible time by advancement in system enabling synergistically combining goodness of modern and/ or alternative therapies in a single unit.

US20130061178A1 discloses a device for providing a meditative experience wherein the device utilizes an animated production that is generally 10 minutes in length and that focuses on the activation of the seven Chakras as a means of providing physical and/or spiritual wellness. The benefits of the device are automatic such that no prior training is required by the user to realize the wellness effect of the device. The user simply needs to view an animated production where the seven rays activate the seven chakras in a specific manner. The production can be viewed on any form of personal display device and can take place anywhere it is desirable to have a meditative experience.

US10737054B1 discloses a system that may include a chamber structure having a substantially oval or “egg” shaped outer shell. The system may also include an interior formed within the outer shell. A user area may be provided in the chamber structure substantially centered front to back and side to side within the outer shell. The system may also include at least one sound emitting device positioned to provide at least one person in the user area with a prescribed sound therapy. The system may also include at least one light emitting device selected to provide the person in the user area with a prescribed light therapy.

US20200078265A1 discloses a Trilogy method provides a process of self-directed energy healing of mind, body, and spirit of a user to release stress and negative issues. More specifically, the trilogy process is a user self-directed therapy/healing process that combines the most effective therapy modalities. Various modalities derived into the trilogy method include unique healing code points, acupuncture points, meridians, chakra energy centers, governing and conception vessels, eye movement, chi, prayer, meditation, releasing statements, and/or any other viable and suitable modalities that encourage physical, mental, and/or spiritual healing for the user. Additionally, the trilogy method incorporates several self-directed hand/palm movements including tapping, rubbing, pressurizing, and stimulating actions. The trilogy method comprises a three-minute self-directed procedure that uses the most effective modalities. Further, the trilogy method can be supplemented with additional procedures derived from effective but optional modalities that total to a five-minute complete trilogy process.

As detailed above, various solutions have been provided according to the existing arts, but all these solutions still have existing challenges because of the limited applications due to the inefficient treatment or therapies provided by the conventional system. At the same time, these therapies are too complex and may not suit everyone. The systems or apparatus provided are not modular. They are mostly dedicated to accommodating a single person suffering from a particular condition and require operators or therapists to take them through the healing experiences. The methods being provided in the prior arts do not target holistic wellness and focuses mainly on providing therapy to individuals. The light, sound, colour, and vibrations being used and how they are employed in the prior arts are way too ordinary and have limited applications.

It is, therefore, important to work on the alternative solution to develop a modular improved system that is not a therapy and at the same, obviates the complexity and challenges of the prior arts and provide an innovative system capable of maintaining the holistic wellness of human beings by targeting the entire body.

### SUMMARY OF THE INVENTION

The various objectives and embodiments of the present invention as presented herein are understood to be illustrative and not restrictive and are non-limiting with respect to the scope of the invention.

It is an objective of the present invention to provide a system and method which is capable of providing a sensory experience that de-stresses the user by realigning the body, mind, and soul.

It is an objective of the present invention to provide a system and method which does not solve the health problems through some therapy or treatment but by just using lights, sounds, and vibrations.

It is an objective of the present invention to provide a system and method where people need not do any kind of preparation, instead, they can just sit and sense the lights, sounds, and vibrations being produced inside the system.

A phrase “vicious cycle of stress” has been used in various embodiments, paragraphs, and claims of the present invention, which can be explained as a repetitive cycle through which the human body undergoes due to the regular functioning of the fear-based memory cells elicited by stress (both pain or non-pain related). It can be further explained by the generation of response due to stress that catalyzes the secretion of cortisol, that further results in maladaptive or negative response which could enable an individual to feel catastrophizing, ruminating, or being helpless causing the prolonged or excessive activation of HPA (Hypothalamic-Pituitary-Adrenal) axis resulting in cortisol dysfunction accompanied by inflammation, these all altogether ultimately lead to pain and depression. There are always fear-based memory cells associated with the above process, so whenever the body experiences any kind of stress, the response is picked up by the fear-based memory cells that may result in pain and depression. Every time the body experiences stress, it undergoes the generation of negative response, activation of HPA axis followed by cortisol dysfunction and inflammation, this is the cycle that repeatedly runs in the human body, which is called a vicious cycle of stress. The objective of the present invention is to eliminate this cycle from the human body and render them free from any ailments, if any, by providing a sensory experience that destresses and realigns the body, mind, and soul.

A term “holistic state” has been used in various embodiments, paragraphs, and claims of the present invention, which refers to a particular duration inside the system or duration for a particular kind of sensory experience or duration of sensitizing one or more organs/chakras altogether or any kind of mental state at any point of time while the method is performed, and all the components of the system are operational. Also, CONTENT projecting means should be considered equivalent to the light projecting means.

In accordance with one embodiment of the present invention, there is provided a method for improving holistic health of humans comprising allowing one or more individuals to accommodate in an open or closed framework, producing a combination of a plurality of elements by synergising sounds, lights, colours, vibrations, and visual sights, and showering the said combination of a plurality of elements upon the one or more individuals for healing the holistic wellness and wherein the method comprises of synergising the combination of a plurality of elements to showering upon the one or more individuals to enable them sequentially transitioning from one holistic state to the another synergistically for healing the holistic wellness by eliminating the existing vicious cycle of stress in the one or more individuals, wherein, transitioning comprises of overlapping of each holistic state of the human body with the next holistic state corresponding to the combination of the plurality of elements.

The term “desirable response” has been used in various embodiments, paragraphs, and claims of the present invention, which includes but is not limited to how different organs of the body respond or elicit reactions within the body corresponding to absorbing the plurality of elements, or how different set of organs are impacted sequentially or one by one by activating them for a particular condition. It may include the release of certain hormones, repairment of body cells, generation of nerve impulses for hormonal and biochemical processes inside the body. The responses from the individual could be physical, sensory, cognitive, or emotional by stimulation with the help of the plurality of elements.

The term “dome” has been used in various embodiments, paragraphs, and claims of the present invention, which is an inverted hemisphere structurally and modularly developed as per the sacred geometry.

The term “chakra” has been used in various embodiments, paragraphs, and claims of the present invention, which corresponds to a bundle of nerves, major organs, and various other energy points or areas in our body responsible for balancing the well-being of humans.

In accordance with one exemplary embodiment of the present invention, there is provided a method for improving holistic health of humans in an open or closed framework comprising harmonizing and equilibrating the mind and body organs by synergising the sound frequencies, light, and spectral wavelengths employing a combination of a plurality of elements capable of simultaneously producing responsive healing frequencies and wavelengths for showering upon the one or more individuals to bring healing effects in their health, wherein said method comprises:
a) reviving the memory by projecting the synergised sound and light wave spectrum over visual sights to activate the dormant visual zones of brain for chakra alignment;
b) recognizing the health-related problem by orchestrating the synergised sound and light wave spectrum over the users enabling them to communicate with their inner essence and accessing the existing problem;
c) bringing the user’s mental and physical state into a required healing mode for healing the particular recognized health-related problem by showering the combination of a plurality of elements;
d) activating the body organs by vibrating the vital points of the user’s body at vibratory healing frequency and showering the combination of a plurality of elements to elicit a desirable response;
e) resetting and re-energising the mental and physical state by re-aligning the organs based on the subsequent elicited desirable responses by showering the combination of a plurality of elements; and
f) re-orienting the subsequent elicited response for self-actualizing the healing of the holistic health by showering the combination of a plurality of elements; and
wherein said method in the above-mentioned sequence (a-f) comprises of synergising the combination of a plurality of elements to generate energy vortex and energy fields to shower upon the one or more individuals to enable them sequentially transitioning from one holistic state to the another synergistically for healing the holistic wellness by eliminating the existing vicious cycle of stress in the one or more individuals, wherein, transitioning comprises of overlapping of each holistic state with the next holistic state corresponding to the combination of the plurality of elements.

In accordance with the above exemplary embodiment of the present invention, wherein said plurality of elements is having an element impacting by penetrating laterally the lights of complementary wavelengths with respect to each eye of the individual to elicit a desirable response within their mind and body.

In accordance with the above exemplary embodiment of the present invention, wherein said plurality of elements is having an element impacting by penetrating the lights of wavelengths in a narrow range in the eyes of the individual in order to elicit a desirable response within their mind and body.

In accordance with the above exemplary embodiment of the present invention, wherein said plurality of elements is having an element impacting by stimulating the individuals with alternating binaural sounds and healing frequencies in order to elicit a desirable response within their mind and body.

In accordance with any of the above embodiment of the present invention, wherein said plurality of elements is having an element impacting by stimulating the individuals by striking with the sound frequencies corresponding to planets, gongs, and chants, etc. to elicit a desirable response within their mind and body.

In accordance with any of the above embodiment of the present invention, wherein said plurality of elements is having an element impacting by stimulating the individuals by the sequential visual sights in combination with geometric patterns altogether representing an anecdote in order to elicit a desirable response within their mind and body.

In accordance with the exemplary embodiment of the present invention, wherein said framework is modularly designed sacredly and geometrically to stimulate the individuals for eliciting a desirable response within their mind and body.

In accordance with any of the above embodiment of the present invention, wherein said plurality of elements is having an element impacting by stimulating the individuals from their sense of olfaction, tactile perception, and gustation in order to elicit a desirable response within their mind and body.

In the present invention, the method starts with making the individuals feel comfortable, safe and acquaint them with the surrounding of the framework. In order to make individuals feel comfortable, safe and secure, elements from nature such as laughter or just simple calm space are used for avoiding any unnecessary and unfavourable disturbances from outside the framework, which help individuals to expel out negative energies from their body. The above step is to make individuals accustomed to the internal surrounding of the framework by blocking any disturbances from outside.

According to the present invention, once the individuals are accommodated inside the safe and secured surrounding within the framework, the individuals are made to undergo a pre-contemplation stage by reviving their memories mainly through the visual sights synergized with the sound frequencies, light, and spectral wavelengths employing a combination of a plurality of elements for some time, wherein the visual sights are projected by the projecting means onto the internal walls of the framework, wherein certain organs corresponding to memory are triggered, which further elicits a response to enable the individual recall or remember the events occurred in the past in sequence or a random manner, wherein said events are relevant to past memories of an individual, which could be a reason behind his/her suffering. The visual sights synergized with the sound frequencies, light, and spectral wavelengths employing a combination of a plurality of elements triggers the various energy centers of the individual to enable the individual to form the visuals of the sequential or random events that occurred in the past. This particular step “reviving the memory” re-tells the individual a story plotted and connected through the sequential or random events that occurred in the past, wherein the visual sights are relatable to the individual’s past memories.

In the present invention, post reviving the individual’s memory, it is important to recognize the ailments by recognizing the root cause of the individual’s problem by identifying what is triggered corresponding to the revived memory or which part of the body or body area is responding to the revived memory. By orchestrating the combination of the plurality of elements over the users which enables the individuals to communicate with their inner essence and accessing the existing problem and corresponding to the same, one or more body parts respond, and this action is further contemplated to identify the health conditions of each individual. Once the root cause of the problem and the health conditions of the individuals are identified, the plurality of elements in different combination acts on the body parts responded corresponding to the identified health condition of the individual, that further prepares the body to seek catharsis by providing appropriate biological feed in the responded body parts and other parts by properly regulating and enhancing biochemical and biological activities within the body. Once the catharsis is sought, again, one or more body organs are triggered by a combination of a plurality of elements, wherein the sound of the gong, conches, chants, tuning fork, singing bowls & several frequencies covers the full spectrum of sound, it vibrates all the cells, bones and organs, some more than others, depending upon the pitch of those sounds & lateral chromotherapy acting together enhances its beneficial effects, wherein the colour therapy is a protruding and metaphysical action that brings equilibrium in health to the mind and body by creating an electrical impulse in the brain, which stimulates hormonal and biochemical processes in the body. All this enables the individuals to start transitioning into a state in which they are inflicted upon by deep relaxation and at the same time, they feel an influx of energy developing in their body for healing and rejuvenation. Post relaxing and energizing the individuals, there takes place a repetitive showering of said combination of a plurality of elements upon the one or more individuals for re-orienting and orchestrating the induced responses in the body for eliminating the existing vicious cycle of stress in the one or more individuals and post the re-orientation, the repetitive showering of the said combination of a plurality of elements upon the one or more individuals helps in realigning and maintaining all the impacted organs or chakras to enable the mind and body continue functioning both internally and externally in a state which provides deep relaxation and full of energy and renders the individuals devoid of any stress. Further, upon providing a prolonged and repetitive exposure of the combination of a plurality of elements for a definite period of time, the mind and body become used to the same functioning and comes in regular practice, and the fear-based memory cells adapt themselves to continue functioning at a greater activity with better biochemical and biological activity, thus disabling to follow the traditional vicious cycle of stress. All the above steps being performed in continuance gives one or more individuals a sense of acknowledgment and positive outlook for the future by eliminating the old functioning or patterns or fear-based memories and consolidating the new functioning of the mind and body altogether at the end.

In accordance with one of the embodiments of the present invention, the method comprises of combining healing sound frequencies, wavelengths, and rhythm employing the combination of a plurality of elements, channelizing a flow of energy through the mind-body by adjusting the chemical composition of our internal states and regulating brain-hemisphere imbalances, contributing to a natural abatement of fear and despair-emotions that underlie both of these common afflictions.

In accordance with the above embodiment of the present invention, the method balances the nervous system and regulates the chronic stress and tension that is the norm for many people in today’s hyper-stimulated lifestyle, and it also balances the endocrine system to normalize hormone production to balance one’s mood and overall sense of well-being. The vibrations fuel the vital points on the body thus helping to improve blood circulation and get rid of toxins. It acts as a blood pressure regulatory aid as it creates more endorphins in our body and thus helps to create a better mood by stimulating the limbic system of the brain and help individuals who are depressed. There are many areas of the brain that are stimulated when proper frequency hits on that part including the amygdala, orbitofrontal cortex, hippocampus, anterior cingulate gyrus, insula, parahippocampal gyrus, orbitofrontal cortex, and thalamus. The vibrations of each said frequency are capable to rearrange a human’s molecular structure.

### BRIEF DESCRIPTION OF THE DRAWINGS

FIG. 1 shows the latitudinal cross-sectional view of the system for improving holistic health of the humans.

FIG. 2 shows the longitudinal cross-sectional view of the system for improving holistic wellness of the humans.

FIG. 3 shows the top cross-sectional view of the system for improving holistic wellness of the humans.

FIG. 4 shows the front view of the upper portion of the multipurpose integrated apparatus (11).

FIG. 5 shows the top view of the multipurpose integrated apparatus (11).

### DETAILED DESCRIPTION OF THE INVENTION

In accordance with one embodiment of the present invention, there is provided a system for improving holistic health of humans comprises a modular enclosure accommodating one or more individuals for improving their holistic health comprises a plurality of equipment capable of altogether producing a combination of plurality of elements, wherein the plurality of equipment comprises of, a plurality of light and colour producing means fitted inside the enclosure for showering upon the individuals with a required frequency and wavelength in order to elicit a desirable response within their mind and body, and a multipurpose integrated apparatus centrally and vertically located inside the framework, wherein said apparatus is housed with sound and vibration producing means for sensitizing the individuals at a required frequency in order to elicit a desirable response within their mind and body, and one or more interior walls embodying the modular enclosure which is adapted to form visual sights on its inner surface based on the projected light rays from at least one light projecting means, and wherein said plurality of equipment relatively placed inside the framework so as to synergise the produced wavelengths, frequencies and visual sights employing the combination of plurality of elements impacting the one or more individuals in order to enable them sequentially transitioning from one holistic state to the another synergistically for healing the holistic wellness by eliminating the existing vicious cycle of stress in the one or more individuals.

In accordance with the above embodiment of the present invention, wherein said modular enclosure is constructed employing sacred geometry, provided with the plurality of light and colour producing means inside it and encapsulated by an exterior framework.

In accordance with the above embodiment of the present invention, wherein said multipurpose integrated apparatus is provided with one or more reflectors and the at least one light projecting means, wherein the light projecting means is movable and capable of projecting spectrum of light anywhere inside the framework.

In accordance with one of the above embodiments of the present invention, wherein said one or more reflectors positioned relative to the positions of the light and colour producing means, and sound and vibration producing means for reflecting the light and sound at a required wavelength and frequency in the required direction for eliciting a desirable response within individual’s mind and body, wherein said reflectors are two in number, pyramidal in shape, positioned to face each other and preferably, one centrally hanging from the ceiling of the enclosure and other is centrally based on the floor.

In accordance with one of the above embodiments of the present invention, wherein said plurality of light and colour producing means, and one or more sound and vibration producing means are relatively placed so as to optimally shower the one or more individuals with the required wavelengths and frequencies for eliciting a desirable response within their mind and body.

In accordance with one of the above embodiments of the present invention, wherein the plurality of equipment comprises of means for inducing sense of olfaction, gustation and tactile perception, and wherein said plurality of equipment are interchangeably placed depending on the kind of healing to be carried for the one or more individuals and wherein said visual sights formed on the inner surface of the interior walls by projecting light rays which are also incorporated with geometric patterns and are intended to create an anecdote, which when perceived, elicits a desirable response within individual’s mind and body.

In accordance with one of the above embodiments of the present invention, wherein another vibrating means is situated in close proximity to the one or more individuals, wherein the one or more sound producing means produces the sound at frequencies corresponding to the frequencies produced by gongs, chants, planets, alternating binaural sounds.

In accordance with one exemplary embodiments of the present invention, wherein the produced light and colour from the light and colour means are configured to laterally penetrate the eyes of the one or more individuals with complementary wavelength.

In accordance with one exemplary embodiments of the present invention, wherein the produced light and colour from the light and colour means are configured to penetrate the eyes of the one or more individuals in a range of narrow wavelengths.

In accordance with one embodiment of the present invention, there is provided a system for improving holistic health of humans comprising an exterior framework providing the required shape to the system and a modular enclosure encapsulated within the exterior framework and adapted to accommodate one or more individuals, wherein said modular enclosure comprises of a plurality of equipment capable of altogether producing a combination of plurality of elements, wherein the plurality of equipment comprises of a plurality of light and colour producing means fitted inside the enclosure for showering upon the individuals with a required frequency and wavelength in order to elicit a desirable response within their mind and body, a multipurpose integrated apparatus centrally and vertically located inside the framework, wherein said apparatus is housed with sound and vibration producing means for sensitizing the individuals at a required frequency in order to elicit a desirable response within their mind and body, and one or more interior walls embodying the modular enclosure which is adapted to form visual sights on its inner surface based on the projected light rays from at least one light projecting means, and wherein said plurality of equipment relatively placed inside the framework so as to synergise the produced wavelengths, frequencies and visual sights employing the combination of plurality of elements impacting the one or more individuals in order to enable them sequentially transitioning from one holistic state to the another synergistically for healing the holistic wellness by eliminating the existing vicious cycle of stress in the one or more individuals.

In accordance with just above embodiment of the present invention, wherein said required shape to the system can be modified by changing the shape of exterior framework and adapted to encapsulate the interior enclosure, preferably the shape to the system provided by the exterior framework is dome shape. Also, the method disclosed in the above embodiments for improving the holistic wellness can even be performed without including the exterior framework of the system. The exterior framework is installed only when the disclosed method is to be performed in an outer environment where there is a lot of disturbances from outside, otherwise there is no requirement of exterior framework for carrying out the above method in a closed environment.

In accordance with one embodiment of the present invention, there is provided a multipurpose integrated apparatus for producing and projecting lights, colours and sounds comprises of a frame supported by a shaft from top and housed with a multiple light projecting means at its periphery and light producing means between centre and the periphery, a multiple sound producing means located above the frame and around the shaft and at least one reflector affixed on the bottom of the frame adapted to reflect lights.

In accordance with one embodiment of the present invention, there is provided an audio-visual means capable of producing sound baths, frequency vibrations of sound and light and light showers in the framework’s geometric space, wherein, said geometric space embodies the audio-visual means to create a scientific ecosystem enabling human beings to receive a holistic experience for their well-being. The framework is provided in a shape of dome, and contains audio-visual means comprising of light producing means, a pair of pyramidal light reflector housing a plurality of sound producing means and pluralities of seating surfaces.

In accordance with above embodiment of the present invention, the exterior framework is provided with a required heating, ventilation and air-conditioning by placing a HVAC (Heating, Ventilation and Air-Conditioning) equipment on the outer periphery of the exterior framework, wherein said physical structures including HVAC equipment are configured to be transposable, interchangeable and portable.

In accordance with above embodiment of the present invention, wherein, for the purpose of maximizing the resourcefulness and utilization to materiality, said dome is sustainable developed using minimal materials, wherein the same materials are used interchangeably in different shapes and forms throughout the framework.

In accordance with the above embodiment of the present invention, said system is capable of getting configured for easy scalability by using a modularly designed physical structures that further allows the easy expansion of the framework depending on the application, environmental needs, and number of people for accommodation, wherein, modularly designed physical structures are the compatible parts of the system that allows ease of assembly, disassembly, transportation and portability.

In accordance with one exemplary embodiment of the present invention, the smallest (XS) sized enclosure accommodating individuals is 12 feet in diameter and this type of design economizes space and is modularized to serve the purpose in homes.

In accordance with one exemplary embodiment of the present invention, the small (S) sized enclosure accommodating individuals is of 24 feet in diameter and this type of design is modularized for the world and can be used in public parks or recreational spaces or any public area which has easy access or any offbeat place which may add to the beauty to the metropolitan area.

In accordance with one exemplary embodiment of the present invention, the MEDIUM(M) sized enclosure accommodating individuals is 36 feet in diameter and this type of design is modularized for the world and can be used where there are more people like a public event etc. adding beauty to the area, providing homage to the vastness of the human spirit and rewarding spatial and immersion experiences at a community level.

In accordance with one exemplary embodiment of the present invention, the Large (L) sized enclosure accommodating individuals is 48 feet in diameter and this type of design is modularized for the world and can be used where there are more people than a public event, like a social gathering event, etc. which can act as a mob modifier for developing spirituality with physical and emotional improvement.

In accordance with one exemplary embodiment of the present invention, the largest (XL) sized enclosure accommodating individuals is 72 feet in diameter and this type of design is modularized for the world and can be used where there are people in masses and take all of them through the immersive sensory experience that destresses the user, and realigns the body, mind and soul.

In accordance with above embodiment of the present invention, there is provided a system and method for improving holistic wellness in humans, wherein, each of the light source is capable of emitting multiple beam of colour at required wavelength and frequency in a specified pattern for a predetermined time depending on various cultures and working efficiency as a stimulant, wherein, frequencies of each of the sound source or sound producing means varies from 6 Hz 0.5 Hz to 963 Hz) and higher frequencies are also used in some cases, and wavelengths of each of the light source varies from 320-760 nm.

In accordance with one exemplary embodiment of the present invention, there is provided a system and method for improving holistic wellness in humans, wherein, said dome shaped covering comprises of an exterior geodesic dome and interior projection dome, wherein, exterior geodesic dome establishes the shape of the ceiling and provides structural support and interior projection dome functions as an expandable projection screen till infinity.

In accordance with one exemplary embodiment of the present invention, there is provided a system and method for improving holistic wellness in humans, wherein, said pair of pyramidal light reflector is capable of receiving various colour of lights at in a definite range of wavelength and frequency and projecting the same after reflecting the different colours of light at the required wavelength and frequency onto the interior projection dome screen to form visual sights or videos.

In accordance with one exemplary embodiment of the present invention, there is provided a system and method for improving holistic wellness in humans, wherein, said pair of pyramidal light reflector is housed with plurality of sound clusters, which further produces sounds, vibrations in coordination with the visual images or videos being formed onto the interior projection dome screen, wherein, produced sounds, vibrations and formed visuals or videos altogether produces a synergistic effect in terms of affecting the neural and endocrine system of human beings sitting inside the system.

In accordance with the above embodiment of the present invention, there is provided a system and method for improving holistic wellness in humans, wherein, said pair of pyramidal light reflector is configured to hang from the central top of the interior projection dome, wherein, lights of different wavelength and frequencies strikes the pair of pyramidal light reflector angularly, which in return reflects the light and projects onto the interior projection screen.

In accordance with one exemplary embodiment of the present invention, there is provided a system and method for improving holistic wellness in humans, wherein said dome shape covering comprises of two layers as an innermost layer and outermost layer, wherein, said outermost layer forms the outer covering of the dome with two opposite facing passages, said innermost layer is having 2 passages, wherein at least two of the passages of innermost layer superimposes with the 2 passages of outermost layer.

In accordance with one exemplary embodiment of the present invention, there is provided a system and method for improving holistic wellness in humans, wherein the pair of pyramidal light reflector is capable of emitting and reflecting or deflecting colours of light at required wavelengths and frequencies, and project on the interior projection screen after interfering with the light colours produced by light sources placed on the periphery of the system, wherein interference of lights causes the wavelength, frequencies and intensities to result into an effective value in order to form the visual sights along with healing frequencies and wavelength on the interior projection screen.

In accordance with one preferred embodiment of the present invention, wherein one of the said pyramidal light reflector is placed at the bottom centre and other hangs down from central top of the audio visual system, wherein both the pyramidal light reflector oppositely faces each other, wherein each such pyramidal light reflector has at least four converging reflecting surfaces which is capable of bouncing any beam of light back and forth between the interior geodesic projection screen and pair of pyramidal light reflector for an indefinite number of times.

In accordance with one preferred embodiment of the present invention, wherein one of the said pyramidal light reflector is placed at the bottom centre and other hangs down from central top of the audio visual system, wherein both the pyramidal light reflector oppositely faces each other, wherein each such pyramidal light reflector has a minimum of 4 and a maximum of 6 converging reflecting surfaces that creates vortex of reflections between the pyramidal light reflectors by reflecting lights received directly from the light sources or reflected lights from the interior surfaces.

In accordance with one preferred embodiment of the present invention, wherein one of the said pyramidal light reflector is placed at the bottom centre and other hangs down from central top of the system, wherein both the pyramidal light reflector oppositely faces each other, wherein said centrally bottom placed pyramidal light reflector is having at least 4 converging sides and said hanging pyramidal light reflector is having at least 6 converging reflecting surfaces or vice versa, which is capable of bouncing any beam of light back and forth between the interior geodesic projection screen and pair of pyramidal light reflector for an indefinite number of times.

In accordance with one embodiment of the present invention, wherein said pyramidal light reflector is made up of required metals, its armature is made up of wood having a film of mirror coating helping in the reflection for infinite times with converging surfaces to create a very powerful visual effect on the interior geodesic projecting screen.

In accordance with one preferred embodiment of the present invention, wherein outer dimension of said pyramidal light reflector is 6 inches and the incident light emitted from multiple light projecting means makes an angle of 45° with the reflecting surfaces of pyramidal light reflector to create a powerful visual effect on the interior geodesic projecting screen and shower upon the individuals to supplement the holistic experience inside the system.

In accordance with one embodiment of the present invention, said sound clusters produces sounds and vibrations, wherein the rhythm of such sounds and vibrations are automatically played in coordination with formed visual sights on the interior dome projection screen or the visual sights on the interior dome projection screen are picturised in coordination with sounds and vibrations produced from sound clusters.

In accordance with above embodiment of the present invention, said sound clusters produces sounds and vibrations that covers the full spectrum of sound and also vibrates all the cells, organs of human body, depending upon the pitch of those sounds, vibrations and along with the colours or visual and video being formed on projection screen, help in achieving equilibrium in health to our mind and body by generating an electric impulse in our brain, therefore, further stimulating hormonal and biochemical processes in our body.

In accordance with the above embodiment of the present invention, the peculiar combination of sounds, vibrations, images and video visuals in a specified pattern is made to bombard on human beings sitting inside the dome covering for a definite period of time, wherein said peculiar combination act as a stimulant capable of bringing physiological and psychological change in humans for holistic wellness.

In accordance with one exemplary embodiment of the present invention, wherein, the light emitted from the light showers after getting reflected or deflected from pyramidal light reflector at a predetermined wavelength followed by projecting on the interior geodesic dome, wherein the light coming off the interior projecting screen of a specific wavelengths enters into the eyes of humans sitting inside the system and further exhibits Simultaneous Adjacent Chroma Stimulus (SACS) to influence the balance of human’s brain hemisphere by penetrating wavelengths of complimentary colours into each half of the human’s visual field, thereby exerting a differential influence on each of the two brain hemispheres to re-balance the autonomous nervous system.

In accordance with just above embodiment of the present invention, wherein, the light emitted from the light showers after getting reflected or deflected from pyramidal light reflector at a predetermined wavelength followed by projecting on the interior geodesic dome, wherein the light coming off the interior projecting screen of a specific wavelengths enters into the eyes of humans sitting inside the system and further exhibits super chroma, wherein, while exhibiting the super chroma, said light coming off the interior projecting screen entering into the eyes of humans sitting inside the system comprises of a narrow band of wavelengths of at least 10 nm (single colour), without any interference with other colours to influence the balance of human’s brain hemisphere by penetrating the narrow band of wavelengths of complimentary colours into each half of the human’s visual field, thereby exerting a differential influence on each of the two brain hemispheres to re-balance the autonomous nervous system.

The term “Simultaneous Adjacent Chroma Stimulus(SACS)” and “Super Chroma” have been used in the above exemplary embodiments of the present invention, wherein said SACS is defined as a phenomena by which the light enters into the human eyes, wherein each of the eye receives complimentary colour of light at complimentary wavelengths with respect to each other to balance the autonomous nervous system, wherein said “Super Chroma” is defined as a phenomena by which the single light comprising a very narrow band of wavelengths enters into the human eyes to balance the autonomous nervous system.

According to the present invention, the system is capable of adjusting the chemical composition of human’s internal states and regulating brain-hemisphere imbalances, it normalizes hormone production by balancing the endocrine system and provides overall sense of well-being. The vibrations fuel the vital points on our body thus helping to improve the blood circulation and get rid of toxins by re-arranging the molecular structure human’s molecular structure.

According to the present invention, there is provided an audio visual system that having sound baths, light showers and pyramidal light reflector, wherein said sound baths, light showers and pyramidal light reflector interact among each other in a synergistic manner to produce sound and vibrations and form visual sights with the help of projecting means and interference of lights in a specified pattern on to the interior projection screen for a definite period of time displaying an anecdote based on the culture of human beings sitting inside the dome shaped system, wherein said anecdote acts as an stimulant for human beings for reviving memory by adjusting the chemical composition of human’s internal states and regulating brain-hemisphere imbalances, it normalizes hormone production by balancing the endocrine system and provides overall sense of well-being. The vibrations fuel the vital points on our body thus helping to improve the blood circulation and get rid of toxins by re-arranging the molecular structure human’s molecular structure.

According to the present invention, the method for improving holistic wellness also serve as a as nerve tonier, pain reliever, motor stimulant, regulating body temperature and biological cycles including sleep, also, bombarding a proper frequency upon human beings affects the digestive system by boosting up the metabolic system, increases energy levels. The exposure of the pattern formed by the system is capable of controlling depression on menopause, spleen, sedative & work as a muscle relaxant, lymphatic relaxant with the benefits of building up leucocytes in the body thus enhancing all the cellular activity. By sitting inside the system for improving the holistic wellness and experiencing an optimized sounds, vibrations, visual images and videos, altogether, in coordination with each other, also helps in solving problems such as headaches, the proper vibration in human’s body produces Nitric oxide which is a molecule that the body produces to help its 50 trillion cells communicate with each other by transmitting signals throughout the entire body. The positive impact of optimum frequency with light waves is also incorporated into the system, that further stimulate acupuncture and trigger points and increases circulation in areas being stimulated, such as wounds when past the acute stage. The exposure to audio-visual experience inside the system has a triggering effect on the lymph system which reduces many critical physical ailments and also help in triggering secretion of neurochemicals in the brain which can heal all those problems which are associated with the limbic system in this audio-visual environment, one can experience a rise in the endorphin level & also creates hypoactive cellular activity, such as non-union fractures and chronic splints activates humoral and endocrine functions.

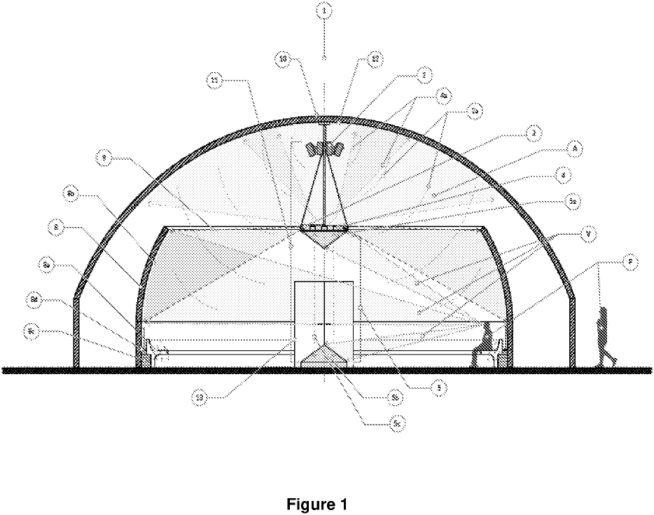

Referring to FIG. 1, a full cross-sectional latitudinal view of the system (1) for improving holistic health of humans is shown, wherein the system (1) comprises of an exterior framework 10 providing a dome shape to the system. An enclosure (9) is provided within the exterior framework (10), wherein the enclosure (9) comprises of two interior walls (8) embodying the structure of the enclosure (9), wherein said each interior wall (8) is having seating surfaces (8a) along the wall (8) to accommodate one or more individuals at the bottom raised up by a certain height from the ground, a projecting screen (8b) above the seating surfaces (8a), a plurality of vibrating means (8c) under the seating surfaces (8a) and a light producing means (8d) fitted just behind the seating surfaces (8c), wherein the light producing means (8d) is positioned so as to incident lights laterally upwards on to the projecting screen (8b). There is provided a multipurpose integrated apparatus (11) centrally and vertically located inside the framework (10) within the enclosure (9) and hanging from the ceiling (12) of the framework (10), wherein said multipurpose integrated apparatus (11) comprises of a plurality of sound producing means (2) directed downwardly to produce alternating binaural sounds (2a), a plurality of light projecting means (3) configured to form visual sights on to the projecting screen (8b), another light producing means (4) configured to incident lights upwardly on to the ceiling of the dome (10) and two reflectors (5) - upper reflector (5a) and lower reflector (5b) in square pyramidal shape, wherein the upper reflector (5a) is affixed just below the light projecting means (3) and another light producing means (4), and both upper (5a) and lower reflector(5b) are positioned to face each other, the upper reflector (5a) is facing downwardly and said lower reflector(5b) is facing upwardly, wherein said reflectors (5) are relatively placed so as to create a vortex of reflection (5c) and deviate the reflected light from the ceiling (12) of the dome (10) and the projecting screen (8b) towards the individuals (P) sitting on the seating surfaces (8a), wherein said exterior framework (10) is only required when the system is to be installed in an outer environment. There is also provided a pair of door (13) at the ends of interior walls for entry and exit of the individuals (P).

At any time inside the enclosure (9) when the all the equipment are operational, individuals (P) experiences a spatial visual resonance (V) created by visual sights formed on the projecting screens (8b) by projecting lights from the light projecting means (3), and light producing means (8d), and by deviating light reflections from the dome (10) and the reflectors (5) towards the individuals for exhibiting Simultaneous Adjacent Chroma Stimulus (SACS), wherein, said SACS received by the eyes and the formed visual sights on the projecting screens are synergised together to enable the individuals (P) getting sensitized at healing wavelengths so as to elicit a desirable response in them.

At any time inside the enclosure (9) when the all the equipment are operational, individuals (P) experiences a spatial aural resonance (A) created by the produced alternating binaural sounds (2a) by the sound producing means (2) directed downwardly towards the individuals (P) and produced vibrations by the plurality of vibrating means (8c) from under the seating surfaces (8a), wherein said alternating binaural sounds (2a) and said vibrations experienced by the individuals are synergised together to enable the individuals (P) getting sensitized at healing frequencies so as to elicit a desirable response in them.

At any time inside the enclosure (9) when all the equipment are operational, individuals (P) experiences both the spatial visual resonance (1) and spatial aural resonance (2) by synergising the healing frequencies and wavelengths for generating energy vortex and energy fields and shower upon the individuals in order to enable them sequentially transitioning from one holistic state to the another synergistically for healing the holistic wellness by eliminating the existing vicious cycle of stress in the one or more individuals, wherein, transitioning comprises of overlapping of each holistic state with the next holistic state through the occurrence of spatial visual resonance and spatial aural resonance.

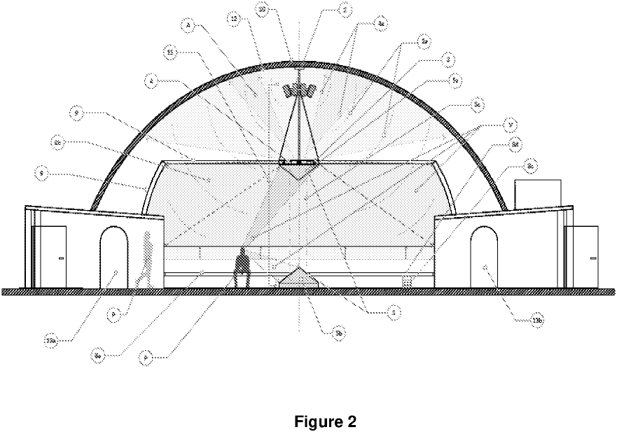

Referring to FIG. 2, a full cross-sectional longitudinal view of the system for improving holistic health of humans is shown, wherein the system comprises of an exterior framework 10 providing a dome shape to the system. An enclosure (9) is provided within the exterior framework (10), wherein the enclosure (9) comprises of an interior wall (8) embodying the structure of the enclosure (9), wherein said each interior wall (8) is having seating surfaces (8a) along the wall (8) to accommodate one or more individuals at the bottom raised up by a certain height from the ground, a projecting screen (8b) above the seating surfaces (8a) and above the heads of individuals (P), a plurality of vibrating means (8c) under the seating surfaces (8a) and a light producing means (8d) fitted just behind the seating surfaces (8c), wherein the light producing means (8d) is positioned so as to incident lights laterally upwards on to the projecting screen (8b). There is provided a multipurpose integrated apparatus (11) centrally and vertically located inside the framework (10) within the enclosure (9) and hanging from the ceiling (12) of the framework (10) laterally on the front side of the interior wall from the top, wherein said multipurpose integrated apparatus (11) comprises of a plurality of sound producing means (2) directed downwardly to produce alternating binaural sounds (2a) going towards the individuals, a plurality of light projecting means (3) configured to form visual sights on to the projecting screen (8b) by projecting lights on it from top to bottom, another light producing means (4) configured to incident lights (4a) upwardly on to the ceiling of the dome (10) and two reflectors (5) - upper reflector (5a) and lower reflector (5b) in square pyramidal shape, wherein the upper reflector (5a) is affixed just below the light projecting means (3) and another light producing means (4), and both upper (5a) and lower reflector(5b) are positioned to face each other, the upper reflector (5a) is facing downwardly and said lower reflector(5b) is facing upwardly, wherein said reflectors (5) are relatively placed so as to create a vortex of reflection (5c) and deviate the reflected light from the ceiling (12) of the dome (10) and the projecting screen (8b) towards the individuals (P) sitting on the seating surfaces (8a), wherein said exterior framework (10) is only required when the system is to be installed in an outer environment. There is also provided a pair of door (13) at the ends of interior wall for entry and exit of the individuals (P), wherein the individuals are supposed to enter and sit on the seating surfaces of the interior wall (8) of one side of the enclosure (9) and face the projecting screen (8b) of the interior wall (8) on the other side of the enclosure (9), wherein said individuals experiences spatial visual and aural resonance (V and A) by synergising healing frequencies and wavelengths and showering upon the individuals as explained in the description of FIG. 1.

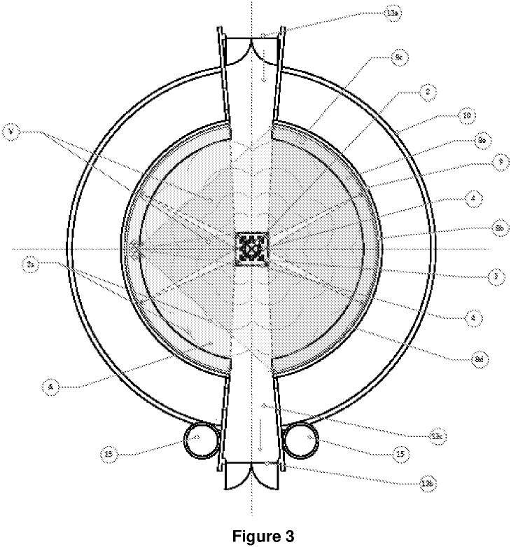

Referring to FIG. 3, a full cross-sectional top view of the system for improving holistic health of humans is shown, wherein the system comprises of an exterior framework (10) forming the outer boundary of the system, an interior enclosure (9) is encapsulated within the exterior framework (10), wherein the interior enclosure (9) is having walls (8) provided with the seating surfaces to accommodate individuals (P). There are openings (13d) being provided at the ends of the walls (8) for providing passages (13c) via entry (13a) and exit door (13b) through the exterior framework (10), wherein a heat, ventilation and air conditioning unit (15) are placed on the outer part of the exterior framework (10) at the doors (13). The integrated multipurpose apparatus centrally located, hanging from top and having a frame (14) that comprises of a plurality of light projecting means (3), plurality of the another light producing means (4) and a plurality of sound producing means (2) so as to the project the lights along the walls (8) and produce alternating binaural sounds (2a) downwardly towards the individuals (P) sitting on the seating surfaces (8a). The Spatial Visual Resonance (V) is created in the sight of view of the individual (P) by reflecting lights from the ceiling (12) of the exterior framework (10) and the reflectors (5). Also, the spatial aural resonance (A) is created by synergising sounds produced from sound producing means (2) and vibrating means (8c)

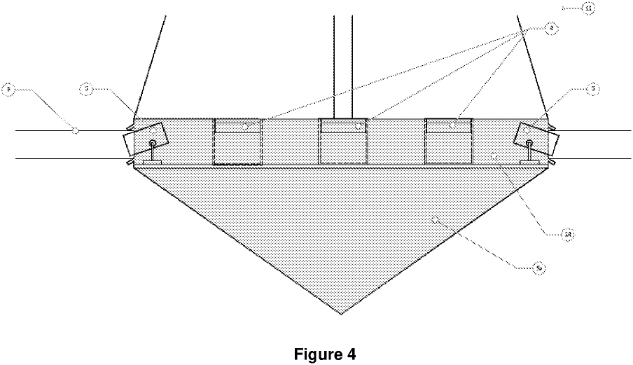

Referring to FIG. 4, the front view of the upper portion below the sound producing means (2) of the multipurpose integrated apparatus (11) of the system for improving holistic health of humans is shown, wherein there is a frame (14) suspended and supported from the top and based in the ceiling (12) of the enclosure (9), wherein said frame (14) comprises of movable light projecting means (3) on its side so as to project lights downwardly and angularly on to the entire projecting screens (8b), and another light producing means (4) configured to incident lights (4a) upwardly on to the ceiling of the dome so as to ultimately reflect the lights from the dome (10) and the reflectors (5) and deviate the reflected lights towards the individuals (P), wherein the lights emitted from the projecting means (3) and the another light producing means (4), when with or without getting reflected reaches the individuals (P), said individuals (P) experiences the spatial visual resonance (V) enabling them eliciting the desirable response in them.

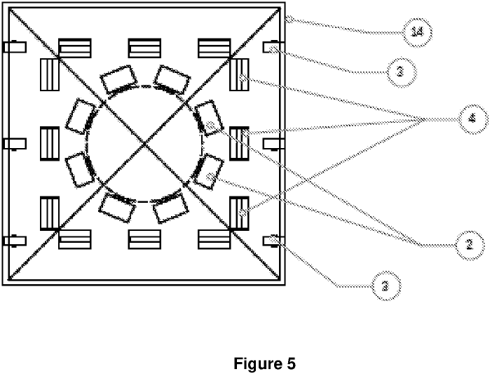

Referring to FIG. 5, the top view of the integrated multipurpose apparatus (11) is shown, wherein it comprises of a pair of sound producing means (2) on and above each side of the frame (14) for producing the alternating binaural sounds in the downward direction uniformly in all directions towards the individuals (P), wherein said frame (14) comprises of three light projecting means (3) on each side of the frame (14) in order to project light beams on to the projecting screens (8b) and the another three light producing means (4) upwardly directed on any two opposite sides of the frame that emits light on to the dome (10) and get reflected back towards the individuals (P) via reflectors (5), wherein said light producing means (4) are capable of producing 13 colours from LED.

Below are the few examples that illustrates the experiences of one or more individuals undergone through the disclosed method performed in the disclosed system:

### Example 1

The experiences of individuals who have undergone the method inside the system are characterized by an understanding that the sum of the steps provides a profound sense of interconnection to the whole and featuring the mind, body, heart, or soul, a wider perspective addressing both the individual and the collective. The individuals are transformed by awakening and nourishing their mind and body with the help of visuals, sound, and light, the system disclosed is like a greenhouse for the individuals. Once the individuals are awakened, they can embody more of the higher transcendent states of being. Usually lower consciousness keeps them in duality, judgement, and discord. Being human, there will be dips up and down but once awakened to higher levels of being, one can redirect out of lower moments by choosing more evolved behaviour. While the method is performed, individuals are invited ascension to higher consciousness by using displays that speak to this level of mind, such as the shimmering infinity mirror, offering communication beyond words, allowing for an “Aha” moment to a deeper structure of the brain that is beyond words. The infinity mirror, along with other displays, offers an invitation to enter a terrain beyond where the ordinary brain can take them. The method enables individuals to experience a more elevated levels of cognizance.

### Example 2

The experiences of individuals who have undergone the method inside the system are characterized by self-actualization, or functioning at the highest transpersonal level, wherein the human body seeks to embrace the heart and the glory, feel a balance of the worries of regular day to day existence and to give genuine feelings of serenity comprehensively. One’s brain, heart, and respiratory rates are controlled, along with the pain reflex & restore emotional balance and mental sanity. It has been seen that individuals develop imagination, ability to dream and get hypnotized, meditate, and becoming emotional and excited and going in trances, etc. Also, increased activity is observed against anxiety, behavioural activation, and inhibition.

In the present invention, the parameter such as angle of incident light with pyramidal reflector, dimensions of pyramidal light reflector and of the entire system, range of light wavelength and frequency of sound waves, placement of different components of the system, arrangement of persons sitting inside the system and materials used in the construction of the system are discussed based on the exemplary embodiments, and may be varied depending on the requirement of users, and may be further worked upon within the scope the disclosure.

Referring the accompanying drawings are provided herein illustrating the further understanding of the present disclosure and are incorporated in and constitute a part of this provisional specification. The drawings illustrate exemplary embodiments of the present disclosure and, together with the description, however, the same will be further expanded within the scope of the disclosure and serve to explain the principles of the present invention.

We claim:

1. A method for improving holistic health of humans in an open or closed framework comprising:
harmonizing and equilibrating the mind and body organs by synergising the sound frequencies, light and spectral wavelengths employing a combination of a plurality of elements capable of simultaneously producing responsive healing frequencies and wavelengths for showering upon the one or more individuals to bring healing effects in their health, wherein said method comprises:
a) reviving the memory by projecting the synergised sound and light wave spectrum over visual sights to activate the dormant visual zones of brain for chakra alignment;
b) recognizing the health-related problem by orchestrating the synergised sound and light wave spectrum over the users enabling to communicate with their inner essence and accessing the existing problem;
c) bringing the user’s mental and physical state into a required healing mode for healing the particular recognized health related problem by showering the combination of a plurality of elements;
d) activating the body organs by vibrating the vital points of the user’s body at vibratory healing frequency and showering the combination of a plurality of elements to elicit a desirable response;
e) resetting and re-energising the mental and physical state by re-aligning the organs based on the subsequent elicited desirable responses by showering the combination of a plurality of elements; and
f) re-orienting the subsequent elicited response for self-actualizing the healing of the holistic health by showering the combination of a plurality of elements; and
wherein said method in the above-mentioned sequence (a-f) comprises of synergising the combination of plurality of elements to generate energy vortex and energy fields so as to showering upon the one or more individuals in order to enable them sequentially transitioning from one holistic state to the another synergistically for healing the holistic wellness by eliminating the existing vicious cycle of stress in the one or more individuals, wherein, transitioning comprises of overlapping of each holistic state with the next holistic state corresponding to the combination of the plurality of elements and;
wherein said method comprises of experiencing a combination of spatial visual resonance and spatial aural resonance, wherein said spatial visual resonance is created by reflecting synergised lights and colours towards the individuals, wherein said spatial aural resonance is created by directing the synergised sounds towards the individuals.

2. The method as claimed in claim 1, wherein said plurality of elements is having an element impacting by penetrating laterally the lights of complementary wavelengths with respect to each eye of the individual in order to elicit a desirable response within their mind and body.

3. The method as claimed in claim 1, wherein said plurality of elements is having an element impacting by penetrating the lights of wavelengths in a narrow range in the eyes of the individual in order to elicit a desirable response within their mind and body.

4. The method as claimed in claim 1, wherein said plurality of elements is having an element impacting by stimulating the individuals with alternating binaural sounds and healing frequencies in order to elicit a desirable response within their mind and body.

5. The method as claimed in claims 1-4, wherein said plurality of elements is having an element impacting by stimulating the individuals by striking with the sound frequencies corresponding to planets, gongs and chants, etc. in order to elicit a desirable response within their mind and body.

6. The method as claimed in claims 1-5, wherein said plurality of elements is having an element impacting by stimulating the individuals by the sequential visual sights in combination with geometric patterns altogether representing an anecdote in order to elicit a desirable response within their mind and body.

7. The method as claimed in claim 1, wherein said framework is modularly designed sacredly and geometrically to stimulate the individuals for eliciting a desirable response within their mind and body.

8. The method as claimed in claim 1-7, wherein said plurality of elements is having an element impacting by stimulating the individuals from their sense of olfaction, tactile perception and gustation in order to elicit a desirable response within their mind and body.

9. A method for improving holistic health of humans comprising:
allowing one or more individuals to accommodate in an open or closed framework;
producing a combination of plurality of elements by synergising sounds, lights, colours, vibrations and visual sights; for showering upon the one or more individuals present inside the framework; and
showering said combination of plurality of elements upon the one or more individuals for healing the holistic wellness; and
wherein the method comprises of synergising the combination of plurality of elements so as to showering upon the one or more individuals in order to enable them sequentially transitioning from one holistic state to the another synergistically for healing the holistic wellness by eliminating the existing vicious cycle of stress in the one or more individuals, wherein, transitioning comprises of overlapping of each holistic state with the next holistic state corresponding to the combination of the plurality of elements; and
wherein said method comprises of experiencing a combination of spatial visual resonance and spatial aural resonance, wherein said spatial visual resonance is created by reflecting synergised lights and colours towards the individuals, wherein said spatial aural resonance is created by directing the synergised sounds towards the individuals.

10. The method as claimed in claim 9, wherein said plurality of elements is having an element impacting by penetrating laterally the lights of complementary wavelengths with respect to each eye of the individual in order to elicit a desirable response within their mind and body.

11. The method as claimed in claim 9, wherein said plurality of elements is having an element impacting by penetrating the lights of wavelengths in a narrow range in the eyes of the individual in order to elicit a desirable response within their mind and body.

12. The method as claimed in claims 9-11, wherein said plurality of elements is having an element impacting by stimulating the individuals with alternating binaural sounds and healing frequencies in order to elicit a desirable response within their mind and body.

13. The method as claimed in claims 9-12, wherein said plurality of elements is having an element impacting by stimulating the individuals by striking with the frequencies of sounds corresponding to planets, gongs and chants, etc. in order to elicit a desirable response within their mind and body.

14. The method as claimed in claims 9-13, wherein, said plurality of elements is having an element impacting by stimulating the individuals by the sequential visual sights in combination with geometric patterns altogether representing an anecdote in order to elicit a desirable response within their mind and body.

15. The method as claimed in claims 9, wherein said framework is modularly designed sacredly and geometrically to stimulate the individuals for eliciting a desirable response within their mind and body.

16. The method as claimed in claims 9-15, wherein said plurality of elements is having an element impacting by stimulating the individuals from their sense of olfaction, tactile perception and gustation in order to elicit a desirable response within their mind and body.

17. A modular enclosure (9) accommodating one or more individuals (P) for improving their holistic health comprises:
a plurality of equipment capable of altogether producing a combination of plurality of stimulating elements;
wherein the plurality of equipment comprises of:
a plurality of light and colour producing means fitted inside the enclosure (9) for showering upon the individuals (P) with a required frequency and wavelength in order to elicit a desirable response within their mind and body; and
a multipurpose audio-visual integrated apparatus (11) centrally and vertically located inside the enclosure (9), wherein said apparatus (11) is housed with sound and vibration producing means (2) for sensitizing the individuals at a required frequency in order to elicit a desirable response within their mind and body, and provided with one or more reflectors (5a and 5b) and at least one light projecting means (3), wherein the at least one light projecting means (3) is movable and capable of projecting spectrum of light anywhere inside the enclosure;
one or more interior walls (8) embodying the modular enclosure (9) which is adapted to form visual sights on its inner surface (8b) based on the projected light rays from at least one light projecting means (3);
wherein said modular enclosure is characterized in that by the plurality of equipment relatively placed inside the enclosure (9) so as to synergise the produced wavelengths, frequencies and visual sights employing the combination of plurality of stimulating elements impacting the one or more individuals in order to enable them sequentially transitioning from one holistic state to the another synergistically for improving the holistic wellness by eliminating the existing vicious cycle of stress in the one or more individuals: and
wherein light and colour producing means (4) and sound producing means (2) emits the wavelength and frequencies and resultantly forms the synergised wavelength and frequencies so as to enable individuals experience a combination of spatial visual resonance (V) and spatial aural resonance (A), wherein said spatial visual resonance (V) is created by reflecting synergised lights and colours towards the individuals(P), and wherein said spatial aural resonance (A) is created by directing the synergised sounds towards the individuals (P).

18. The modular enclosure (9) as claimed in claim 17, wherein said modular enclosure (9) is structurally constructed employing sacred geometry, provided with the plurality of light and colour producing means (4) inside it and encapsulated by an exterior framework (10).

19. The modular enclosure (9) as claimed in claim 19, wherein said one or more reflectors (5a and 5b) positioned relative to the positions of the light and colour producing means (4), and sound (2) and vibration producing means (8c) for reflecting the light and sound at a required wavelength and frequency in the required direction for eliciting a desirable response within individual’s mind and body.

20. The modular enclosure (9) as claimed in claim 19, wherein said reflectors (5a and 5b) are two in number, pyramidal in shape, positioned to face each other and preferably, one centrally hanging from the ceiling (12) of the enclosure (9) and other is centrally based on the floor.

21. The modular enclosure (9) as claimed in claim 17, wherein said plurality of light and colour producing means (4), and one or more sound (2) and vibration producing means (8c) are relatively placed so as to optimally shower the one or more individuals with the required wavelengths and frequencies for eliciting a desirable response within their mind and body.

22. The modular enclosure (9) as claimed in claim 17, wherein said plurality of equipment are interchangeably placed depending on the kind of healing to be carried for the one or more individuals.

23. The modular enclosure (9) as claimed in claim 17, wherein said visual sights formed on the inner surface (8a) of the interior walls (8) by projecting light rays which are also incorporated with geometric patterns and are intended to create an anecdote, which when perceived, elicits a desirable response within individual’s mind and body.

24. The modular enclosure (9) as claimed in claim 17, wherein another vibrating means (8c) is situated in close proximity to the one or more individuals (P).

25. The modular enclosure (9) as claimed in claim 17, wherein the plurality of equipment comprises of means (4) for inducing sense of olfaction, gustation and tactile perception.

26. The modular enclosure (9) as claimed in claim 17, wherein the one or more sound producing means (2) produces the sound at frequencies corresponding to the frequencies produced by gongs, chants, planets, alternating binaural sounds (2a).

27. The modular enclosure (9) as claimed in claim 17, wherein the produced light and colour from the light and colour means (4) are configured to laterally penetrate the eyes of the one or more individuals with complementary wavelength.

28. The modular enclosure (9) as claimed in claim 17, wherein the produced light and colour from the light and colour means (4) are configured to penetrate the eyes of the one or more individuals in a range of narrow wavelengths.

29. The modular enclosure (9) as claimed in claim 17, wherein a method is provided (1) comprising harmonizing and equilibrating the mind and body organs by synergising the sound frequencies, light and spectral wavelengths employing a combination of a plurality of elements capable of simultaneously producing responsive healing frequencies and wavelengths for showering upon the one or more individuals to bring healing effects in their health.

30. The modular enclosure (9) as claimed in claim 30, wherein said method (1) comprises of synergising the combination of plurality of elements so as to showering upon the one or more individuals in order to enable them sequentially transitioning from one holistic state to the another synergistically for healing the holistic wellness by eliminating the existing vicious cycle of stress in the one or more individuals, wherein, transitioning comprises of overlapping of each holistic state with the next holistic state corresponding to the combination of the plurality of elements.

31. A system (1) for improving holistic health of humans comprises:
an exterior framework (10) providing the required shape to the system (1); and
a modular enclosure (9) encapsulated within the exterior framework (10) and adapted to accommodate one or more individuals (P) comprises:
a plurality of equipment capable of altogether producing a combination of plurality of elements;
wherein the plurality of equipment comprises of:
a plurality of light and colour producing means (4) fitted inside the enclosure (9) for showering upon the individuals (P) with a required frequency and wavelength in order to elicit a desirable response within their mind and body;
a multipurpose integrated apparatus (11) centrally and vertically located inside the framework (10), wherein said apparatus (11) is housed with sound and vibration producing means (2) for sensitizing the individuals at a required frequency in order to elicit a desirable response within their mind and body, and provided with one or more reflectors (5a and 5b) and at least one light projecting means (4), wherein the at least one light projecting means (4) is movable and capable of projecting spectrum of light anywhere inside the enclosure (9);
one or more interior walls (8) embodying the modular enclosure (9) which is adapted to form visual sights on its inner surface (8b) based on the projected light rays from at least one light projecting means (3); and
wherein said system is characterized in that by the modular enclosure (9) comprising plurality of equipment relatively placed inside the framework (10) so as to synergise the produced wavelengths, frequencies and visual sights employing the combination of plurality of elements impacting the one or more individuals (P) in order to enable them sequentially transitioning from one holistic state to the another synergistically for healing the holistic wellness by eliminating the existing vicious cycle of stress in the one or more individuals; and
wherein, light and colour producing means (4) and sound producing means (2) emits the wavelength and frequencies and resultantly forms the synergised wavelength and frequencies so as to enable individuals experience a combination of spatial visual resonance (V) and spatial aural resonance (A), wherein said spatial visual resonance (V) is created by reflecting synergised lights and colours towards the individuals, and wherein said spatial aural resonance (A) is created by directing the synergised sounds towards the individuals (P).

32. A multipurpose integrated apparatus (11) for producing and projecting lights, colours and sounds comprises:
a frame (14) supported by a shaft from top and housed with a multiple light projecting means (3) at its periphery and light producing means (3) between centre and the periphery;
a multiple sound producing means (2) located above the frame (14) and around the shaft; and
at least one reflector (5a) affixed on the bottom of the frame (14) adapted to reflect lights.
